(19) United States
(12) Patent Application Publication
(10) Pub. No.: US 2005/0143811 A1
(43) Pub. Date: Jun. 30, 2005

(54) Methods and apparatus for mitral valve repair

(76) Inventor: Fidel Realyvasquez, Palo Cedro, CA (US)

Correspondence Address: HELLER EHRMAN LLP, 275 MIDDLEFIELD ROAD, MENLO PARK, CA, 94025-3506, US

(21) Appl. No.: 11/003,917

(22) Filed: Dec. 2, 2004

Related U.S. Application Data
(60) Provisional application No. 60/526,663, filed on Dec. 2, 2003.

(51) Int. Cl. A61F 2/24

(52) U.S. Cl. 623/2.36; 623/2.11; 623/902

(57) ABSTRACT

Methods and apparatus are provided for valve repair. In one embodiment, the apparatus includes a first bridge portion and a second bridge portion. The apparatus may also include at least one base on each bridge portion. Attachment of the first bridge portion and the second bridge portion brings an anterior leaflet of the valve closer to the posterior leaflet and reduces a gap therebetween.

### CROSS-REFERENCE TO RELATED APPLICATIONS

The present application claims the benefit of priority to co-pending U.S. Provisional Application Ser. No. 60/526,663 (Attorney Docket No. 40450-0003) filed Dec. 2, 2003. This application is incorporated herein by reference for all purposes.

### BACKGROUND OF THE INVENTION

1. Field of the Invention

The invention relates to methods for valve repair and more specifically to mitral valve repair. The invention is particularly useful in the repair and correction of mitral valve regurgitation.

2. Description of Related Art

Essential to normal heart function are four heart valves, which allow blood to pass through the four chambers of the heart in one direction. The valves have either two or three cusps or leaflets, which comprise of fibrous tissue that are attached to the walls of the heart. The cusps open when the blood flow is flowing correctly and then close to form a tight seal to prevent backflow.

The four chambers are known as the right and left atria (upper chambers) and right and left ventricle (lower chambers). The four valves that control blood flow are known as the tricuspid, mitral, pulmonary and aortic valves. In a normal functioning heart, the tricuspid valve allows inflow of deoxygenated blood from the right upper chamber (right atrium) to the right lower chamber (right ventricle). When the right ventricle contracts, the pulmonary valve allows one-way outflow from the right ventricle to the pulmonary vascular bed which carries deoxygenated blood to the lungs. The tricuspid valve is close during this time. The mitral valve, also a one-way inflow valve, allows oxygenated blood, which has returned to the left upper chamber (left atrium), to the lower left chamber (left ventricle). When the left ventricle contracts, the oxygenated blood is pumped through the aortic valve to aorta. During left ventricular ejection of blood the mitral valve is closed. When the ventricle is at the end of its contractile state the aortic valve begins to close and the cardiac cycle repeats itself.

Clinical cardiac decomposition (or heart failure) result from heart valve malfunction, such as mitral insufficiency. Mitral valve insufficiency, also known as mitral regurgitation, is a common cardiac abnormality where the mitral valve leaflets do not completely close when the left ventricle contracts. This allows blood to flow into the left atrium, this results in left ventricular overload and if the condition is not corrected, the added workload will eventually cause left ventricular enlargement and dysfunction resulting in heart failure.

Various approaches to correct mitral valve pathology have included valve replacement, chordae tendinea shortening or replacement and mitral annular repair also known as annuloplasty. Annuloplasty and valvuloplasty procedures have been developed to correct mitral valve insufficiency.

Mitral valve insufficiency typically results from ischemia of the papillary muscles (chronic ischemic mitral regurgitation or CIMR) or connective tissue degeneration of the mitral leaflets or cordae tenedinae. A combination of these factors can coexist in the same patient. Mitral regurgitation can result from a change in the size and shape of the mitral annulus. There is evidence that posterior annulus trends to enlarge to a greater degree than the anterior annulus. This is because the anterior annulus is attached to the strong fibrous skeleton to the heart and the posterior annulus is supported by muscle (a much more elastic tissue).

Various approaches to correct mitral valve pathology have included valve replacement, chordea tendinea shortening or mitral annular repair also known as annuloplasty. Annuloplasty procedures have been developed to correct mitral valve insufficiency. The present method of achieving competence of the regurgitant mitral valve is to perform a mitral valve repair, which frequently requires placement of a mitral annuloplasty ring. Studies have shown that ring annuloplasty abolishes dynamic annular motion and immobilizes the posterior leaflet. Rings of all types used to perform annuloplasty can have an adverse effect on mitral valve function.

A recent concept on mitral valve function has been brought to the surface and has shed light on what may be a more appropriate repair in chronic ischemic mitral regurgitation (CIMR). The concept of Septal Lateral Annular Cinch (SLAC) has many advantages especially in the ischemic mitral valve where the mitral valve mechanism is frequently morphologically normal but dysfunctional. This concept has important advantages: 1) the isolated reduction of mitral septal-diameter can correct CIMR and may potentially simplify mitral valve corrective procedures; 2) SLAC preserves leaflet mobility and does not freeze the posterior leaflet (this frequently converts a bi-leaflet valve to a uni-valve leaflet valve); 3) SLAC preserves physiologic dynamics; and 4) SLAC maintains physiologic mitral annular morphology for proper function. These advantages in combination may play a significant role in remodeling the left ventricular geometry and give the best possible environment for improving the durability of the morphologically normal mitral valve mechanism.

Although Stanford University has done research to validate the concept of SLAC and its efficacy in improving mitral valve performance, there is no technology presently available to perform this procedure in patients. It is becoming apparent that traditionally surgeons have been performing annuloplasty to indirectly achieve SLAC. In trying to achieve SLAC with circumferential or “C” shaped annuloplasty rings (flexible or rigid), however, the end result has been freezing of the posterior leaflet, creation of transvalvular gradient and loss of annular flexibility. These adverse dynamics can result in stresses placed on the repair. Data now suggest that the anterior-posterior dimension is more critical than the commissure-commissure dimension in achieving mitral valve competence and possibly achieving left ventricular remodeling.

Given the rising number of patients with congestive heart failure, there is an opportunity to develop technology to perform SLAC percutaneous in patients with morphologically normal but regurgitant mitral valves.

### SUMMARY OF THE INVENTION

The present invention provides solutions for at least some of the drawbacks discussed above. Specifically, some embodiments of the present invention provide an improved methods for treating various valve ailments. It is one object of the present invention to develop methods and devices to simplify the repair procedure so that more patients can benefit from mitral valve repair. Another object involves achieving SLAC using percutaneous techniques. At least some of these and other objectives described herein will be met by embodiments of the present invention.

In one embodiment, the present invention provides a method of improving valve morphology at a target site. The method comprises providing an apparatus having a first configuration, with a reduced outer diameter and a second configuration, with an expanded outer diameter. The apparatus is advanced into the body in the first configuration. The apparatus is expanded into the second configuration. This typically occurs near the target site. A first portion and a second portion of the apparatus are attached to tissue at the target site, wherein attachment brings an anterior leaflet of the valve closer to the posterior leaflet and reduces a gap therebetween. Penetrating members may be advanced into the tissue wherein the penetrating members may act as fasteners to hold the apparatus in place.

In one embodiment, the apparatus includes a first bridge portion and a second bridge portion. The apparatus may also include at least one base on each bridge portion. Attachment of the first bridge portion and the second bridge portion brings an anterior leaflet of the valve closer to the posterior leaflet and reduces a gap therebetween.

A further understanding of the nature and advantages of the invention will become apparent by reference to the remaining portions of the specification and drawings.

### BRIEF DESCRIPTION OF THE DRAWINGS

FIG. 1 shows a prior art technique for suturing a mitral valve.

FIG. 2 is a cross-sectional view of the heart with one embodiment of the present invention mounted over the mitral valve.

FIG. 3 shows another view of one embodiment of the present invention.

FIGS. 4 and 5 show embodiments of the present invention with different shaped bases.

FIGS. 6 through 8 show various views of yet another embodiment of the present invention.

FIGS. 9 and 10 show various views of another embodiment of the present invention.

FIGS. 11 and 12 show various views of a still further embodiment of the present invention.

FIGS. 13 through 15C show various views of one embodiment of the present invention using three bridge portions.

FIGS. 16 through 19 show various views of another embodiment of the present invention using three bridge portions.

FIG. 20 shows one embodiment of the present invention using only two bridge portions.

FIG. 21 shows one embodiment of a kit according to the present invention.

### DESCRIPTION OF THE SPECIFIC EMBODIMENTS

The present invention provides a solution for treating various valve disorders. Specifically, some embodiments of the present invention provide methods and devices for percutaneous mitral valve repair. For some embodiments of these penetrating member drivers, the invention provides a transvascular solution. At least some of these and other objectives described herein will be met by embodiments of the present invention.

It is to be understood that both the foregoing general description and the following detailed description are exemplary and explanatory only and are not restrictive of the invention, as claimed. It may be noted that, as used in the specification and the appended claims, the singular forms “a”, “an' and “the” include plural referents unless the context clearly dictates otherwise. Thus, for example, reference to “a material” may include mixtures of materials, reference to “an anchor” may include multiple anchors, and the like. References cited herein are hereby incorporated by reference in their entirety, except to the extent that they conflict with teachings explicitly set forth in this specification.

In this specification and in the claims which follow, reference will be made to a number of terms which shall be defined to have the following meanings:

“Optional” or “optionally” means that the subsequently described circumstance may or may not occur, so that the description includes instances where the circumstance occurs and instances where it does not. For example, if a device optionally contains a feature for analyzing a blood sample, this means that the analysis feature may or may not be present, and, thus, the description includes structures wherein a device possesses the analysis feature and structures wherein the analysis feature is not present.

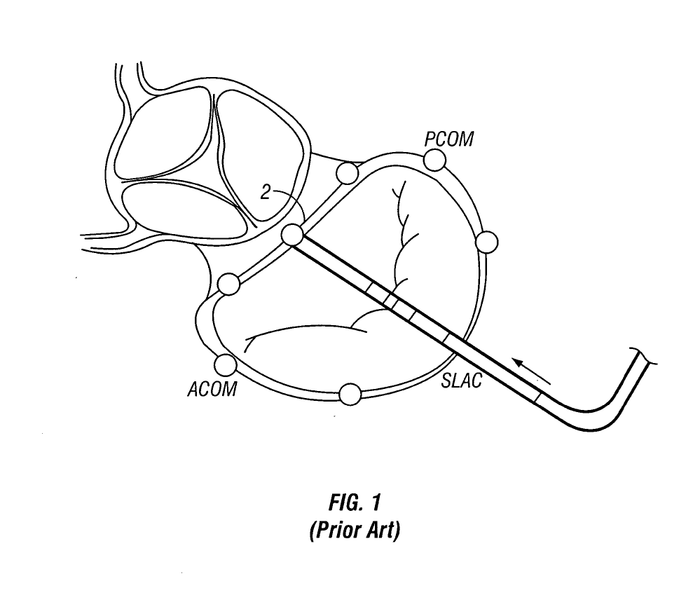

Referring now to FIG. 1, a Septal Lateral Annular Cinch (SLAC) procedure is shown on a mitral valve. Simple septal-lateral annular cinching with sutures may be used to treat acute ischemic mitral regurgitation. The procedure may involve a septal-lateral transannular suture 2 anchored to the midseptal mitral anulus and externalized to a tourniquet through the midlateral mitral anulus and left ventricular wall. This technique is used to reduce annular size, yet allow normal mitral annular dynamic motion.

By way of example and not limitation, the present invention may involve the percutaneous insertion of a mitral valve repair device that is delivered to the left atrium through the inter-atrial septum using catheter-based technology. The device may be used to improve the shape and/or dimensions of the valve and thus improve valve performance. Most percutaneous intra cardiac mitral valve prosthetic devices will initially be delivered with the assistance of cardiopulmonary bypass techniques. Initially, technology such as that available from Heartport, Inc. (Johnson and Johnson, Inc.) will be useful to facilitate deployment of percutaneous devices. Ultimately with development of newer imaging and fastening devices percutaneous SLAC will be performed without cardiopulmonary bypass.

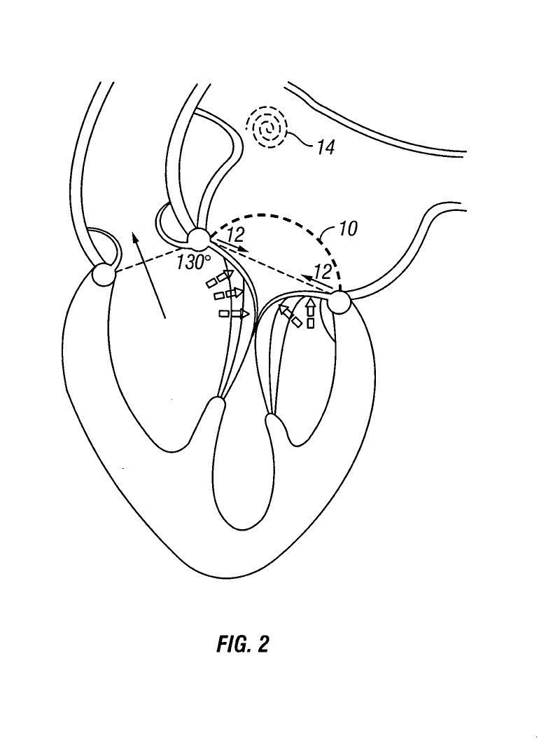

Referring now to FIG. 2, one embodiment of the present invention will now be described. The annular bridge 10 may be used to bring tissue together as indicated by arrows 12. The annular bridge 10 may have a spiraled or curled configuration 14 during delivery and then assume a second expanded configuration as seen in FIG. 2 once the bridge 10 is delivered to the valve site.

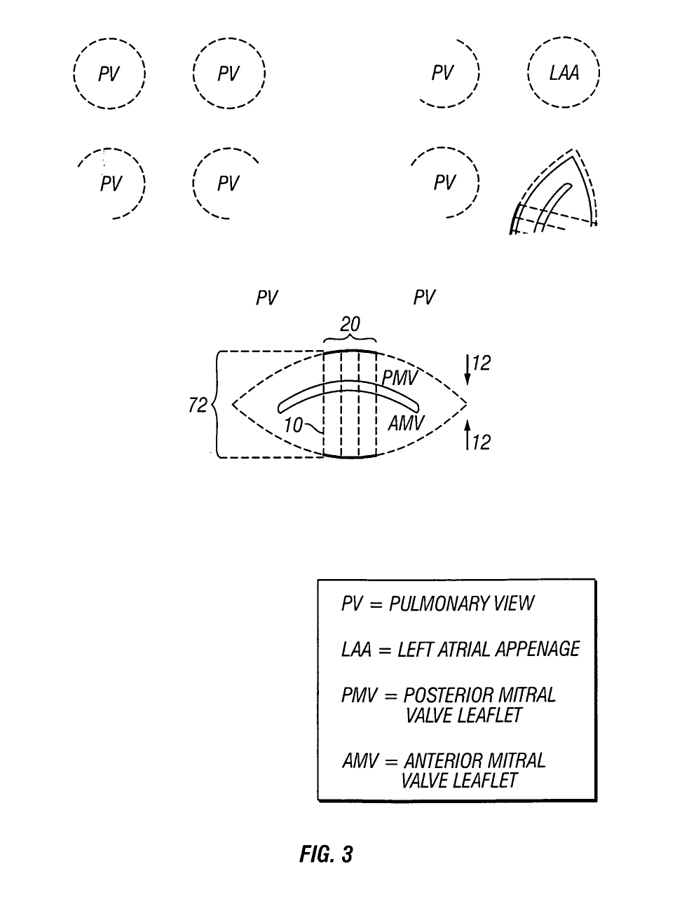

As seen in FIG. 3, this design of device 10 comprises an annular harness with two components: annular 20 and bridge 22 components. This device 10 may be delivered to the left atrium with a catheter it is deployed and unwrapped. When deployed, the bridge component (which may be made of a variety of material including but not limited to semi-rigid nitinol) may be attached to the mitral annulus by needle tines on both sides of the annular bridge. The annular component 20 of the device would be made of flexible material. Once the bridge 10 is attached it would be reinforced by transvascular delivered fasteners. Coaptation would be achieved by pre-determined bridge size, drawing tissue inward as indicated by arrows 12. This design simplicity allows for device deployment using percutaneous intra cardiac techniques without cardiopulmonary bypass.

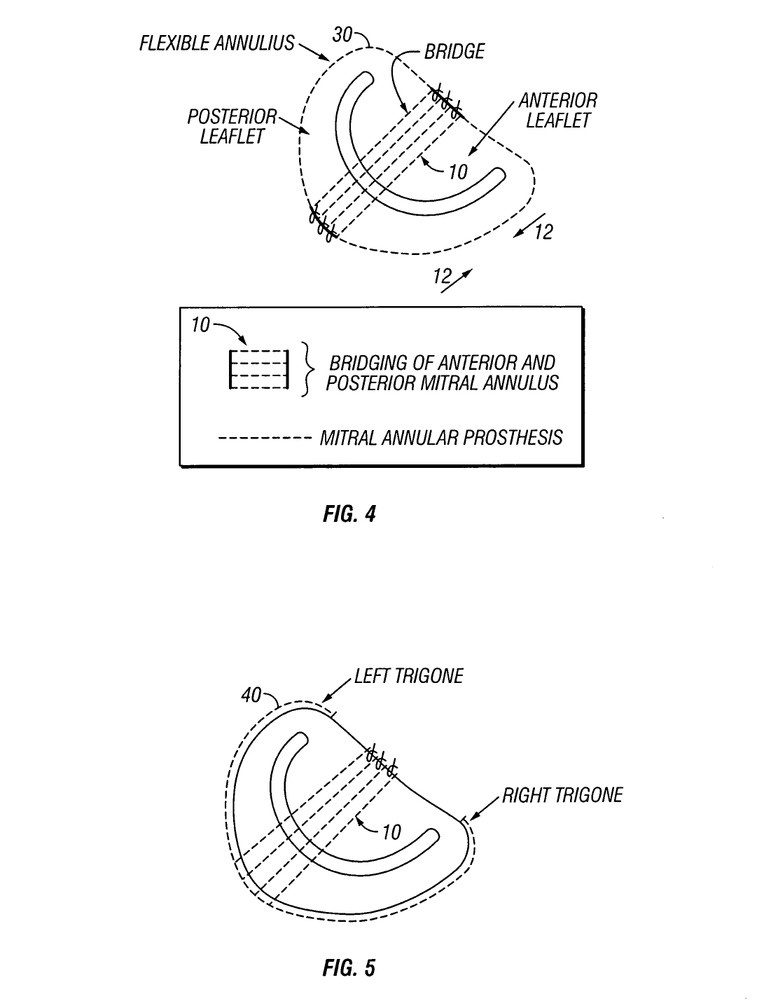

Referring now to FIG. 4, another embodiment of the present invention is shown. There are two possible modifications to the design of FIGS. 2 and 3. One would be to incorporate a circumferential ring prosthesis 30. The ring prosthesis 30 may comprise of flexible nitinol ring with Dacron, mesh, or other clothe covering. The ring prosthesis 30 may be attached by a variety of techniques including but not limited to sutures, preattached sutures and needles, shape memory clips that will engage tissue, anchors, other fastener device, or any combination of the above. Similar techniques may be used to attach the device 10 to the mitral valve site.

Referring now to FIG. 5, a still further embodiment of the present invention is shown. The other would be a “C” ring design where a C-shaped prothesis 40 is attached to the bridge 10. The “C” ring design would have the advantage of less likely to cause left ventricular outflow tract obstruction.

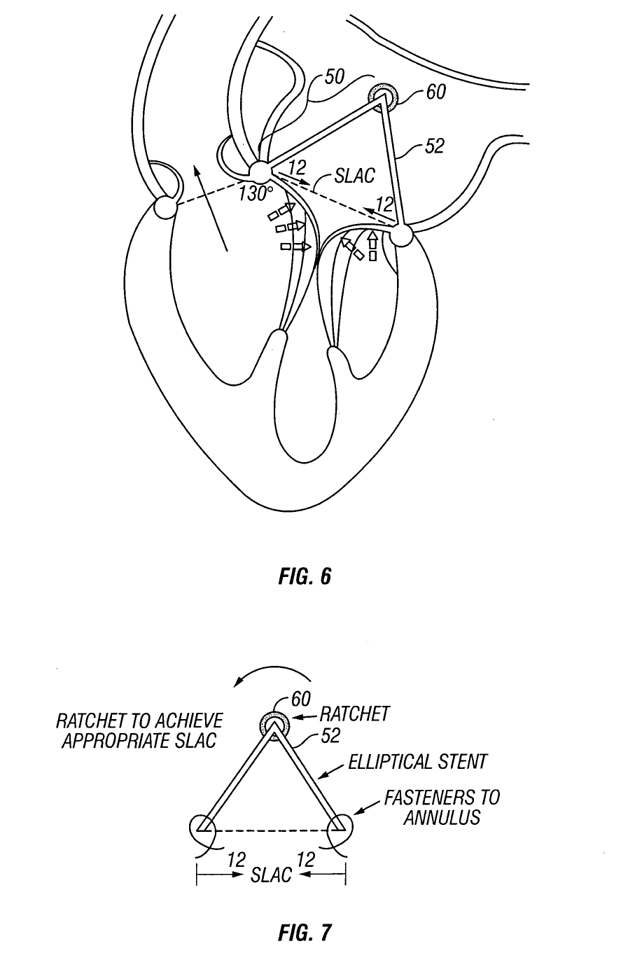

Referring now to FIG. 6, yet another embodiment of the present invention will now be shown. This device 50 may be delivered using percutaneous intravascular catheter techniques. It may be loaded at the end of a specially designed multipurpose catheter. Once the catheter is delivered through the inter-atrial septum, the two wired stents 52 made of nitinol is deployed and allowed to expand. The anterior portion of the stent 52 is attached to the annulus temporarily with the tines anchored to the wire. The posterior portion is anchored to the posterior annulus with similar tines. Once the stent is in proper position, the wires are re-enforced to their position with transvascular delivered fasteners to the posterior and anterior annular attachment points.

Referring now to FIG. 7, when the device 50 has been anchored, the catheter is rotated in a counter clock direction to activate the ratchet mechanism 60 to reduce the dimension between the anchored segments as indicated by arrows 12. The will create the appropriate coaption and achieve mitral valve competence.

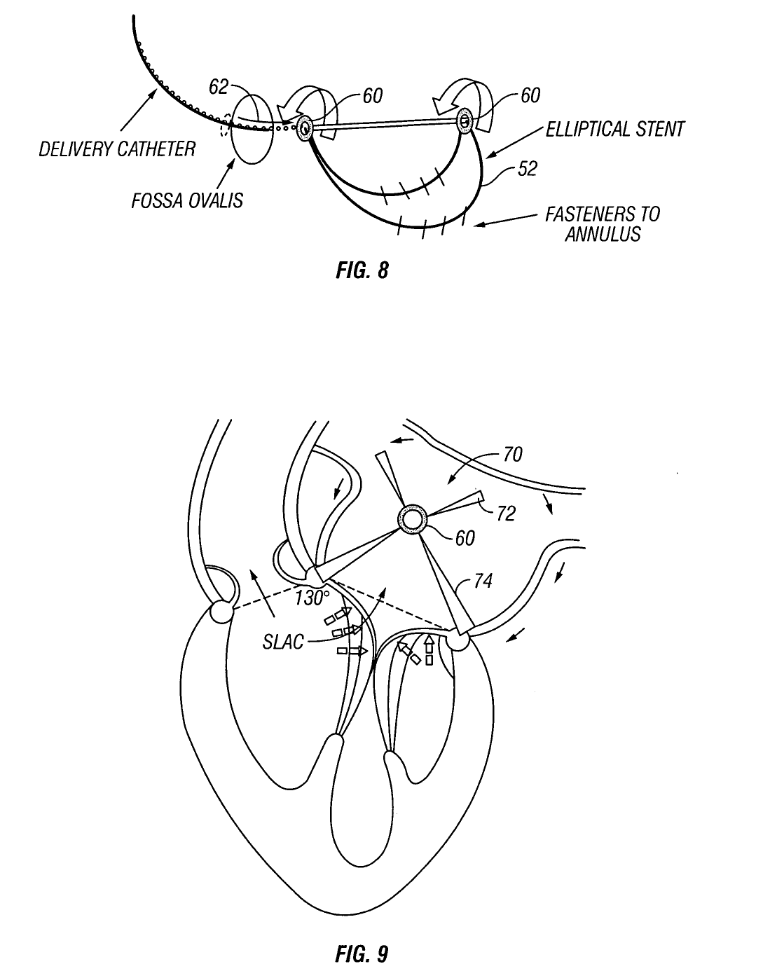

FIG. 8 shows a side view of the device 50 with stents 52 and ratchet 60. In this embodiment, the proximal ratchet 60 may be pushed forward as indicated by arrow 62. This movement forward will case the stents 52 to bow outward and assume the configuration shown in FIG. 8. This moves the device 50 from a first configuration, having a smaller outer diameter that is desirable during delivery, to a second configuration having a bowed configuration shown in FIG. 8 that allows for attachment.

Referring now to FIG. 9, yet another embodiment of the present invention will be described. This device 70 comprises two elements, a mural 72 and an annular. 74 component. The device 70 would be made of nitinol of a specific gauge. The stent components 72 and 74 would be wrapped around a catheter and when it is delivered to the left atrium, it would be deployed and the device 70 unwrapped. The circumferential mural portion 72 of the stent would be attached to the dome of left atrial wall and inferor wall of left atrium, reducing the potential obstruction of the pulmonary veins. The mitral annular component 74 of the stent would be attached to the mitral annulus. The device 70 may be temporarily attach with tines on the annular component of the stent. Once the annular component 74 is attached, trans-vascular fasteners may be used to re-enforce the attached points. The catheter may then be rotated in a counter-clockwise direction to achieve the appropriate anterior-posterior annular coaptation to achieve mitral valve competence. The catheter would be left in place until the trans-thoracic echo can document satisfactory mitral valve function. The catheter may then be disengaged and the atrial septum repaired.

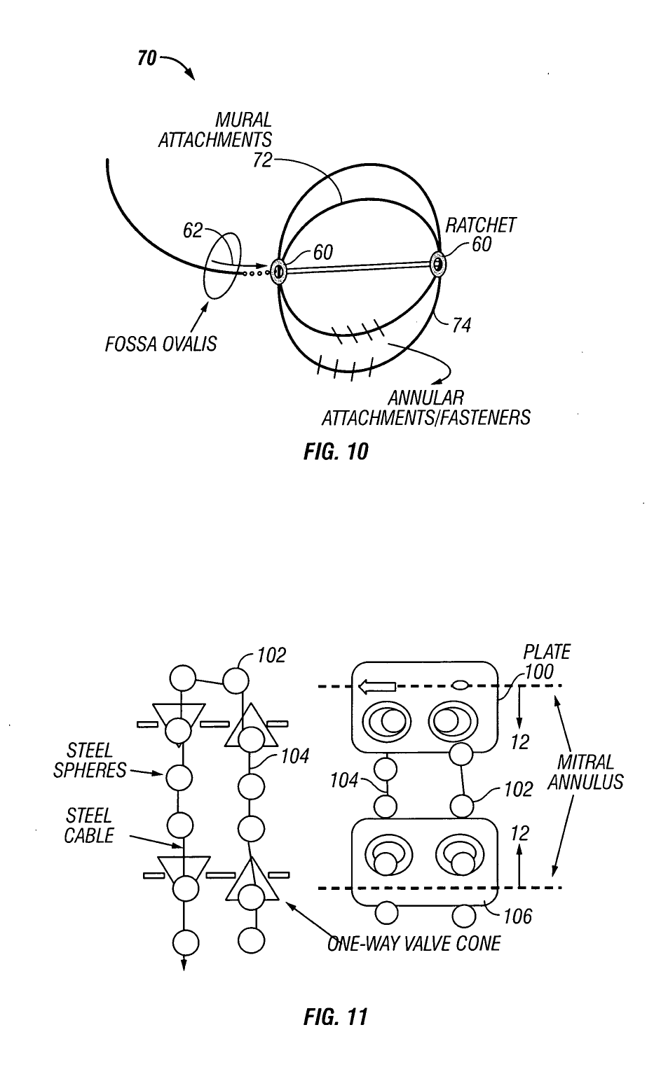

FIG. 10 shows a side view of the device 70 with mural portions 72 and annular portions 74. In this embodiment, the proximal ratchet 60 may be pushed forward as indicated by arrow 62. This movement forward will case the portions 72 and 74 to bow outward and assume the configuration shown in FIG. 8. This moves the device 70 from a first configuration, having a smaller outer diameter that is desirable during delivery, to a second configuration having a bowed configuration shown in FIG. 10 that allows for attachment.

Referring now to FIG. 11, yet another embodiment of the present invention is shown. FIG. 11 shows the concept involved in performing a septal lateral cinch as follows: A plate 100 is attached to the mid anterior mitral annulus into the fibrous skeleton. A variety of techniques including but not limited to suturing, clipping, using shape memory fasteners, or any combination of fasteners may be used to attach the plate. In this nonlimiting example, the plate 100 may be constructed of valve ring Dacron material and may optionally have two one-way valves mechanisms that allow the passage of a series of stainless steel (or proline, plastic, etc.) spheres 102 connected in series by a stainless steel cable 104. A similar plate 106 is at attached to the posterior annulus with the same one-way valve mechanism.

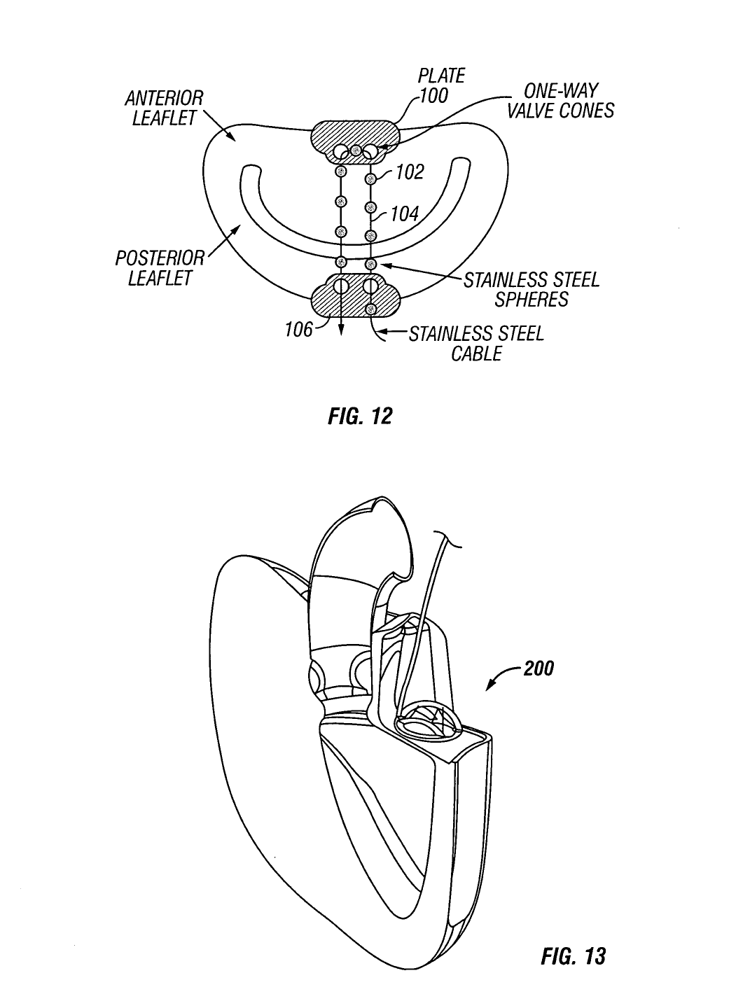

As seen in FIG. 12, with the plates 100 and 102 in place, the string 104 of “pearls” 102 is passed through the one-way valve cone mechanism starting in the posterior plate and strung toward the anterior plate. Once the string of pearls has been advanced from posterior to anterior, the reverse is carried out. The string of pearls is then passed from anterior to posterior through the one-way cone mechanism. It is my impression that very little force for A-P shortening will be necessary to achieve a satisfactory result.

An alternative to the above method is to preload the string of pearls to both the anterior and posterior plates. This alternative would then require only attachment of the anterior and posterior plates. This method would further facilitate and simplify the procedure.

When the string of pearls is in place and the correct amount of shorting has been achieved, the cable attachment to the last pearl is unscrewed for release of the delivery system.

The correct dimensions for achieving SLAC will be determined by calculating the anterior-posterior and the commissure-commissure dimensions by trans-esophageal echocardiography. The appropriate dimension rations will then be calculated.

By filling the heart with blood and encouraging ejection, interventionalist would then pull on the string of pearls to achieve the appropriate A-P dimension. The mitral regurgitation will then resolve as demonstrated by trans-esophageal echocardiography.

The suboptimal results in the surgical treatment of ischemic mitral regurgitation have prompted the development of newer surgical techniques for repair of this condition. Traditionally, surgeons corrected IMR by perfuming mitral valve repair with an annuloplasty ring, flexible or rigid. The concept is to obtain maximal surface contact of the anterior and posterior mitral leaflets by down sizing the mitral annulus. Newer concepts have been proposed to correct IMR. The core concept of these techniques is the reduction of the anterior-posterior mitral annular dimension.

Furthermore, recent experimental data confirm that the aortic and mitral valve functions are interrelated and not independent of one another. From mid-diastole to end-systole, the mitral annulus contracts and the aortic base expands. The net result of this synchrony is facilitated function of both valves trough out the cardiac cycle (Emmanuel Lansac, et. al., J.Thoracic Cardiovasc Surg 2002;123:911-918). In addition, there are deformational dynamics of the aortic root that may be influenced by improper prosthetic selection not only in the aortic position but also the mitral position (Paul Dagum, et.al.,Circulation 1999;100:II-54) Placing a rigid annuloplasty ring on the mitral annulus may restrict normal aortic annular expansion and create stress on the leaflets, the commissures, the sinuses of valsalva and the sinotubular junction predisposing to accelerated structural deterioration of the native aortic valve.

Disruption of the three dimensional mechanical properties of the aortomitral junction can have major clinical implications by selecting the improper prostheses.

Experimental evidence lends credence to the current design of the Mitral Valve Prosthetic Bridge. Embodiments of this device can be used in the repair of the mitral valve to correct mitral regurgitation secondary to myocardial ischemia.

In one embodiment, this device is designed to correct mitral regurgitation by reducing the anterior-posterior annular dimension. It also allows the intertrigonal distance to expand during systole for proper aortic valve function and it allows the native mitral annulus to deform in its physiologic “saddle” shape trough out the cardiac cycle. The added potential advantage is less stress on the native aortic valve structures and durability of the mitral valve repair.

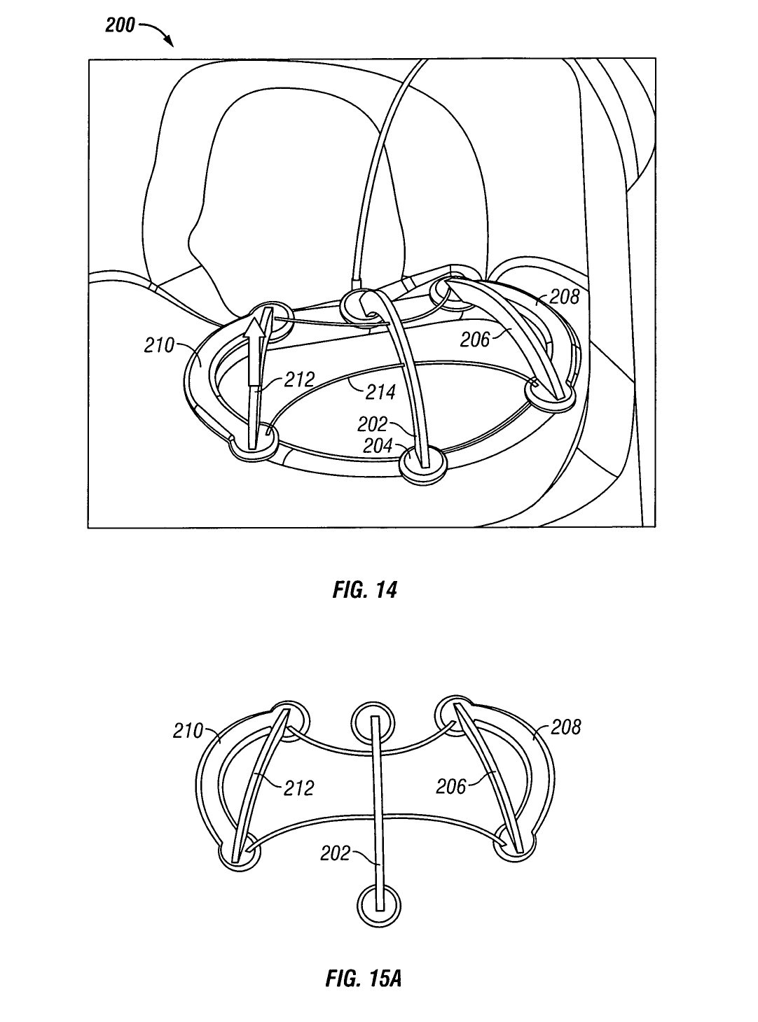

Referring now to FIGS. 13 and 14, one embodiment of the present invention for addressing the above issues will now be described. FIG. 13 shows a device 200 used to changed to valve morphology. In one embodiment, the device 200 is attached at the right and left trigones, anteriorly and the posterior annulus at the junction of P1-P2 and P2-P3. The device would be attached at three contact points lateral to the mid-plane of the mitral valve which would simplify and expedite the time to perform the repair. The device may also be attached the valve between the trigones and on P2.

FIG. 14 more clearly shows the placement of the device 200. The central bridge portion 202 extends across the valve in a trans-annular fashion near the valve center. The portion 202 may have sewing pads 204 coupled to the areas of the bridge portion that will be attached to the valve tissue. A side bridge portion 206 is coupled to a C-shaped or curved sewing pad 208. Another side bridge portion 210 and curved sewing pads 212 may also be used. All three bridge portions 202, 206, and 210 may optionally be coupled together with connectors 214. In addition to bringing the anterior leaflet closer to the posterior leaflet, this linkage by connectors 214 allows the side bridge portions 206 and 210 to stabilize the center bridge portion 202. By way of example and not limitation, the connectors 214 may be wires or rods made of stainless steel, metal, plastic, or a polymer. By way of example and not limitation, the bridge portions 202, 206, and 210 may be made of stainless steel, metal, plastic, or a polymer.

In some embodiments, the bridge portions 202, 206, and 210 may be deployed into the valve separately and attached to the tissue separately. In this embodiment, the connectors 214 may be coupled to the bridge portions after the portions are attached to the valve. Of course, some embodiments may have the bridge portions 202, 206, and 210 coupled to the connectors 214 prior to deployment to the valve. In some embodiments, the device

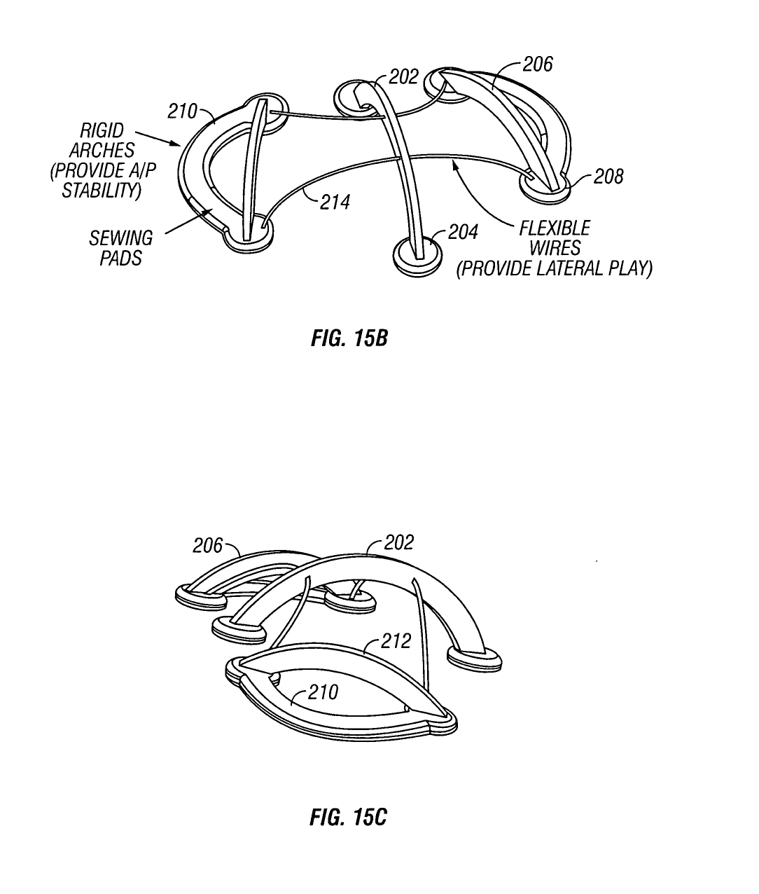

Referring now to FIGS. 1SA to 15C, other views of the device of FIG. 14 are shown. FIG. 15A shows a top down view of the three bridge portions 202, 206, and 210. FIG. 15B shows that the connector 214 may extend from the sewing pad 210 to the bridge portion 214 and then to sewing pad 208. In some embodiments, the connector 214 may extend from bridge portion 206, to portion 202, and to portion 210. In some embodiments, the connectors may extend over the bridge portions connect to the topside surface of the bridge portions. FIG. 15C shows a side view of the bridge portions. As seen in FIG. 15, the side view of the center bridge portion 202 indicates that the portion 202 has a higher arc than those of the side portions 206 and 212. This allows for greater clearance over the center of the valve. Some alternative embodiments may have the arcs at all the same or substantially the same height over the valve. As seen in FIG. 15C, the connector 214 closer to the anterior leaflet may also be concaved or curved in a manner to intersect the center bridge portion 202 at an area further from pad 204 to provide more support to the center bridge while still connected to pads 208 and 210.

As seen in FIGS. 13 to 15C, the sewing pads do not form a ring about the valve annulus. The noncontinuous nature of the sewing pad or support to the bridge portions allows the valve tissue to have the freedom of motion to more effectively regulate flow through the valve. A continuous ring may impede the motion of the valve. The present invention provides gaps between the support or sewing rings that are directly coupled to the valve tissue. These gaps allow for controlled tissue movement. Some embodiments may only have one gap.

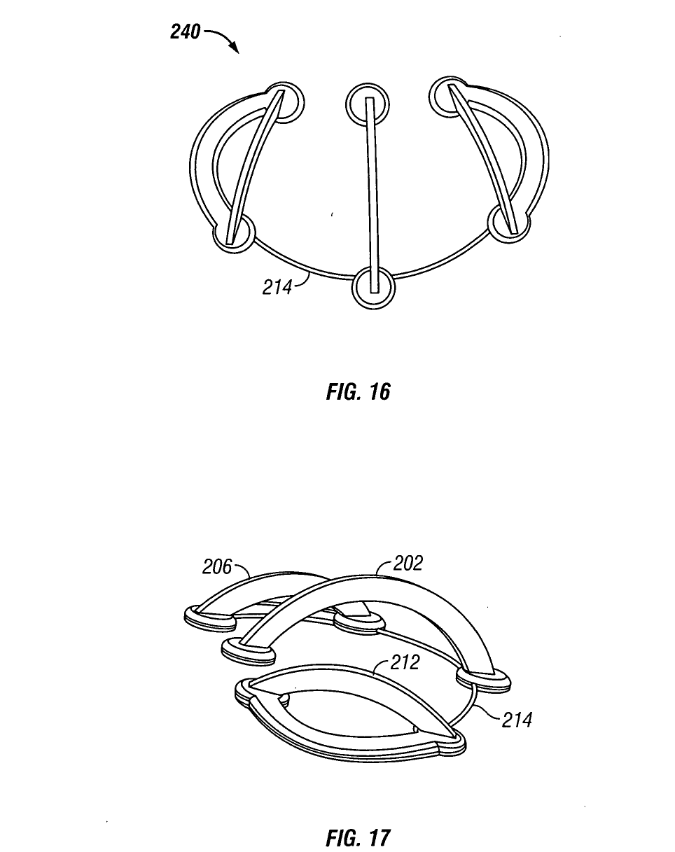

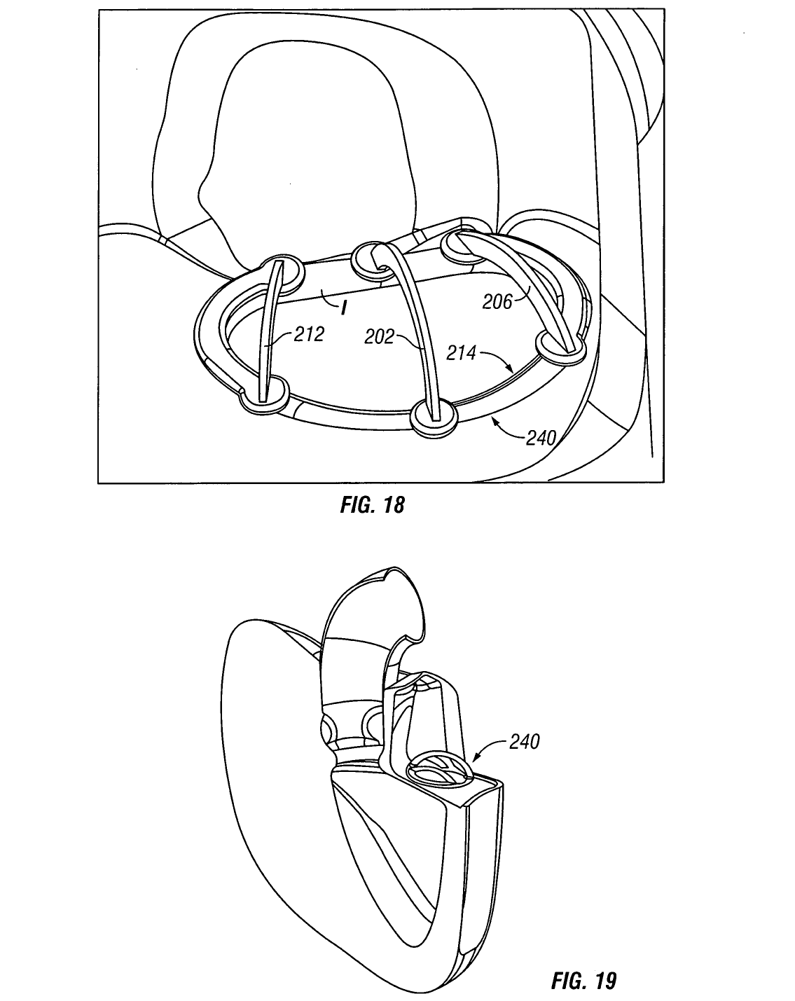

FIGS. 16 and 17 show a still further embodiment of the present invention. In this embodiment, there is only one connector 214 and it extends along a portion of the device 240 that will run along the posterior leaflet. This creates a C-shape base and connects the bridge portions in a location that will not limit the motion of the mitral valve. Specifically, it allows the intertrigonal region I (see FIG. 18) to expand during systole for proper aortic valve function, and it allows the native mitral annulus to deform in its physiologic “saddle” shape trough out the cardiac cycle.

FIG. 17 more clearly shows how the connector 214 run along the base of the portions 202, 206, and 212. Other embodiments may have the connector 214 running higher, connecting to the portions 202, 206, and 212 directly, instead of passing through the base.

FIG. 18 shows the device 240 positioned in the mitral valve to reduce the gap between the anterior leaflet and the posterior leaflet.

FIG. 19 shows a cross-sectional view of the heart with the device 240 positioned over the mitral valve.

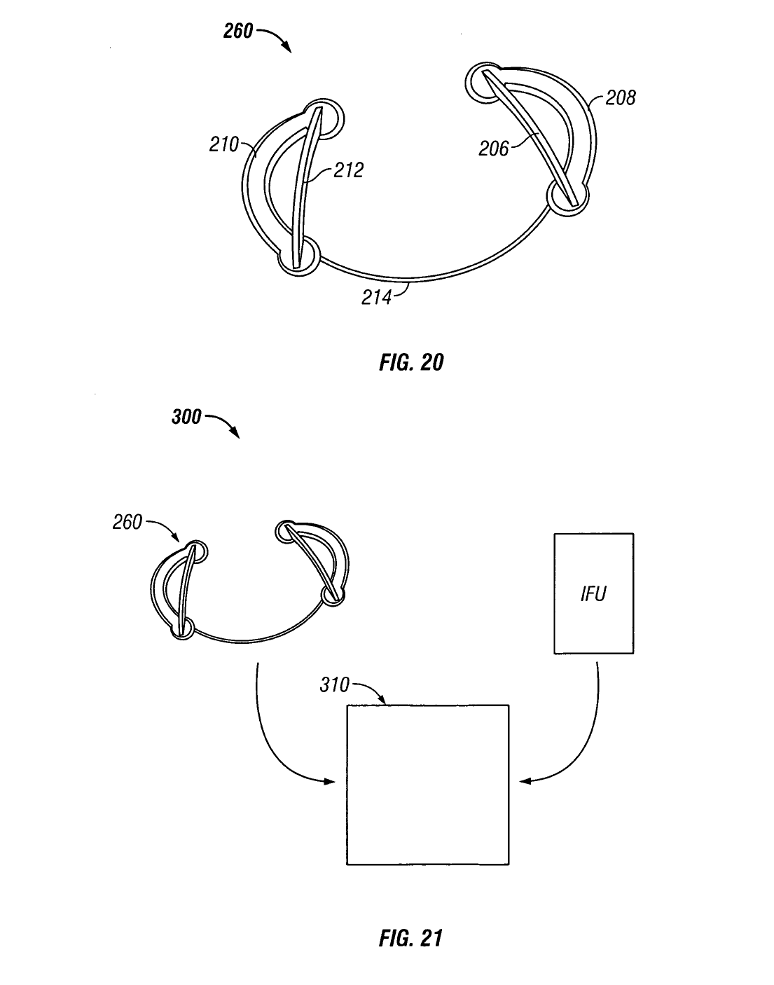

FIG. 20 shows yet another embodiment of the present invention. FIG. 20 shows a device 260 where the center bridge portion 202 is removed and only side portions 206 and 212 remain. A connector 214 may be used to form a C-shaped base with the sewing pads 208 and 210. Other embodiments may have two connectors 214 similar to that used in the embodiment of FIG. 14. It should be understood that in some embodiments, a wider band of material may be used as a connector 214 instead of the wire or rod shown in FIG. 20. Other embodiments may use a C-shaped sewing pad that extend along the entire base and is integrally formed with pads 208 and 210.

FIG. 21 shows one embodiment of a kit according to the present invention. The kit 300 may include a device 260 for improving valve morphology and instructions for use (IFU) setting for the method of attaching the device 260 to the valve. A container 310 of suitable size may be provided to contain the device 260 and the IFU.

While the invention has been described and illustrated with reference to certain particular embodiments thereof, those skilled in the art will appreciate that various adaptations, changes, modifications, substitutions, deletions, or additions of procedures and protocols may be made without departing from the spirit and scope of the invention. For example, with any of the above embodiments, a prosthetic valve or a graft may be premounted on to the apparatus. With any of the above embodiments, the apparatus may be configured to be delivered percutaneously or through open surgery. With any of the embodiments herein, the devices may be attached by a variety of techniques including sutures, preattached sutures and needles, shape memory clips that will engage tissue, anchors, other fastener device, or any combination of the above. With any of the embodiments herein, the number connectors may be increased to greater than 2. Some embodiments may have more than three bridge portions. Some embodiments may only use one bridge portion. With any of the embodiments herein, the user may be provided with bridge portions of a variety of lengths to provide the desired gap reduction between the valve leaflets. In some embodiments, a plurality of bridges may extend across the valve near the center of the valve in a transannular fashion. It should be understood that the present invention may be used on other valves throughout the body.

The publications discussed or cited herein are provided solely for their disclosure prior to the filing date of the present application. Nothing herein is to be construed as an admission that the present invention is not entitled to antedate such publication by virtue of prior invention. Further, the dates of publication provided may be different from the actual publication dates which may need to be independently confirmed. All publications mentioned herein are incorporated herein by reference to disclose and describe the structures and/or methods in connection with which the publications are cited.

Expected variations or differences in the results are contemplated in accordance with the objects and practices of the present invention. It is intended, therefore, that the invention be defined by the scope of the claims which follow and that such claims be interpreted as broadly as is reasonable.

1. A method of improving valve morphology at target a site, the method comprising:
providing an apparatus to improve valve morphology, said device having a first bridge portion configured to extend across the center of the valve in a transannular position when the apparatus is positioned and attached to the valve;
attaching a first base portion and a second base portion of the apparatus to tissue at the target site, wherein attachment brings an anterior leaflet of the valve closer to the posterior leaflet and reduces a gap therebetween; and
advancing penetrating members into said tissue, said penetrating members being secured in said tissue and the apparatus, and act as fasteners.

2. The method of claim 1 further comprising providing at least one other bridge portion coupled to the first bridge portion.

3. The method of claim 1 wherein a gap is defined between the first base portion and the second base portion.

4. The method of claim 1 wherein the first base portion and the second base portion are integrated to form a C-shaped base portion.

5. The method of claim 1 wherein the first base portion and the second base portion are spaced apart.

6. The method of claim 1 wherein the base of each bridge portion is spaced apart from the base of another bridge portions to define gaps to allow the valve tissue to expand therebetween.

7. A method of improving valve morphology at target a site, the method comprising:
providing an apparatus to improve valve morphology, said device having a plurality of bridge portions each configured to extend across the valve in a transannular fashion when the apparatus is positioned and attached to the valve;
attaching a first base portion of one bridge portion and a second base portion another bridge portion to tissue at the target site, wherein attachment brings an anterior leaflet of the valve closer to the posterior leaflet and reduces a gap therebetween; and
advancing penetrating members into said tissue, said penetrating members being secured in said tissue and the apparatus, and act as fasteners.

8. The method of claim 7 further comprising providing at least one other bridge portion coupled to the first bridge portion.

9. The method of claim 7 further comprising a connector coupling the base portions together.

10. The method of claim 7 further comprising a connector coupling the base portions together wherein a gap remains between the base portions and the gap is sized to allow for the intertrigonal region I of the mitral valve to expand during systole for proper aortic valve function.

11. A device for improving valve morphology at target a site, the method comprising:
a bridge portion;
wherein attachment of the bridge portion brings an anterior leaflet of the valve closer to the posterior leaflet and reduces a gap therebetween.

12. A device for improving valve morphology at target a site, the method comprising:
a bridge portion;
a base of the bridge shaped to allow the intertrigonal distance to expand during systole for proper aortic valve function and allow the native mitral annulus to deform in its physiologic “saddle” shape trough out the cardiac cycle;
wherein attachment of the bridge portion brings an anterior leaflet of the valve closer to the posterior leaflet and reduces a gap therebetween.

13. A device for improving valve morphology at target a site, the method comprising:
a first bridge portion;
a second bridge portion;
at least one base on each bridge portion; and
wherein attachment of the first bridge portion and the second bridge portion brings an anterior leaflet of the valve closer to the posterior leaflet and reduces a gap therebetween.

14. The device of claim 13 further comprising a connector connecting first bridge portion to the second bridge portion.

15. The device of claim 13 further comprising a connector coupling the base portions together.

16. The device of claim 13 further comprising a connector coupling the base portions together wherein a gap remains between the base portions and the gap is sized to allow for the intertrigonal region I of the mitral valve to expand during systole for proper aortic valve function.

17. The device of claim 13 further comprising providing at least one other bridge portion coupled to the first bridge portion.

18. The device of claim 13 wherein a gap is defined between the first base portion and the second base portion.

19. The device of claim 13 wherein the first base portion and the second base portion are integrated to form a C-shaped base portion.

20. The device of claim 13 wherein the first base portion and the second base portion are spaced apart.

21. The device of claim 13 wherein the base is a sewing ring.

22. The device of claim 13 wherein the bridge portion is made of metal or stainless steel.

23. The device of claim 13 wherein the bridge portion is rigid in the vertical direction, but flexible in a horizontal direction.

24. The device of claim 13 wherein the bridge portion is rigid about the X axis, but flexible about a vertical Z axis.

25. The device of claim 13 wherein the base of each bridge portion is spaced apart from the base of another bridge portions to define gaps to allow the valve tissue to expand therebetween.

26. A device for improving valve morphology at target a site, the method comprising:
a central bridge portion sized and configured to extend across a center of the valve in a transannular position when the apparatus is positioned and attached to the valve annulus;
at least one side bridge portion;
a C-shaped base portion coupling said central bridge to the side bridge portion;
wherein attachment of the central bridge brings an anterior leaflet of the valve closer to the posterior leaflet and reduces a gap therebetween.

27. A method of improving valve morphology at target a site, the method comprising:
providing an apparatus having a first configuration, wherein the apparatus has a reduced outer diameter and a second configuration, wherein the apparatus has an expanded outer diameter;
advancing said apparatus in the first configuration along a catheter;
expanding said apparatus into said second configuration;
attaching a first portion and a second portion of the apparatus to tissue at the target site, wherein attachment brings an anterior leaflet of the valve closer to the posterior leaflet and reduces a gap therebetween;
advancing penetrating members into said tissue, said penetrating members being secured in said tissue and the apparatus, and act as fasteners.

28. The method of claim 1 wherein said apparatus further comprises using a ratchet to move the first portion and the second portion to reduce the gap between the leaflets.

29. The method of claim 1 wherein said apparatus comprises using an annular ring to stabilize the apparatus against the valve tissue.

30. The method of claim 1 wherein apparatus assumes said second configuration when a proximal ratchet is pushed distally to reduce the distance between the proximal ratchet and a distal ratchet.

31. The method of claim 1 wherein the apparatus is delivered percutaneously.

32. The method of claim 1 wherein attaching comprises delivering a shape memory clip that holds the apparatus to the tissue.
